(19) United States
(12) Patent Application Publication
(10) Pub. No.: US 2026/0357189 A1
(43) Pub. Date: Nov. 12, 2026

(54) METARHIZIUM BRUNNEUM IFST-OT3, DETOXIFIZYME PRODUCED BY SAME, AND USE OF DETOXIFICATION ENZYME

(71) Applicant: INSTITUTE OF FOOD SCIENCE AND TECHNOLOGY, CHINESE ACADEMY OF AGRICULTURAL SCIENCES, Beijing (CN)

(72) Inventors: Fuguo XING, Beijing (CN); Gang WANG, Beijing (CN); Wenqing WU, Beijing (CN); Xu GUO, Beijing (CN)

(21) Appl. No.: 19/138,278

(22) PCT Filed: Nov. 8, 2023
(86) PCT No.: PCT/CN2023/130411

§ 371 Date: Jul. 1, 2025

(30) Foreign Application Priority Data
Dec. 15, 2022 (CN) 202211617717.1

(51) Int. Cl. C12N 1/145; C12N 9/80

(52) U.S. Cl. CPC C12N 1/145; C12N 9/80; C12Y 305/01004

(57) ABSTRACT

Provided are a Metarhizium brunneum strain IFST-OT3, a detoxifizyme produced by same, and use thereof. Provided are a Metarhizium brunneum strain IFST-OT3 (CGMCC Accession No. 40349) with a strong degrading capability against ochratoxin A and B, a detoxifizyme (degrading enzyme) Amh1 produced by same, and a nucleotide sequence encoding the amidase. Additionally, the optimal physicochemical properties of the enzyme, including the influences of temperatures, pH levels, and the presence of metal ions, are provided. The amidase processes an OTA toxin with a substrate concentration of 1 μg/mL in a buffered system at 60 °C and pH 6.0, achieving a degradation rate of up to 100% within 3 minutes. Amh1 has strong adaptability to temperature and acidic pH. The IFST-OT3 strain and the detoxifizyme produced by same can be used for degrading ochratoxin in agricultural products.

### CROSS REFERENCE TO RELATED APPLICATIONS

The present application is a U.S. National Stage application of International Application No. PCT/CN2023/130411, filed on Nov. 8, 2023, which claims priority to Chinese Patent Application No. 2022116177171, filed on Dec. 15, 2022, entitled “Metarhizium brunneum IFST-OT3, detoxifizyme produced by same, and use of detoxification enzyme”, and the entire disclosure of which is incorporated herein by reference in its entirety.

### TECHNICAL FIELD

The present invention relates to the field of microbiology and biological detoxification technology, and in particular to a Metarhizium brunneum strain IFST-OT3 and a detoxification enzyme produced by the same, and uses of the detoxification enzyme.

### BACKGROUND ART

Ochratoxin A (OTA) is a secondary metabolite produced by Aspergillus and Penicillium. In 1993, the International Agency for Research on Cancer classified OTA as a Group 2B carcinogen. In addition, studies have shown that OTA has nephrotoxicity, neurotoxicity, immunotoxicity, and the like (Malir, et al., 2016, Toxins 8:191). Ochratoxin B (OTB) is a precursor of OTA, and its molecular structure has one less chlorine atom than OTA. OTA and OTB can contaminate a variety of agricultural products such as corn, wheat, sorghum, fruits, dairy products, and meat products (Wang, et al., 2022, Frontiers in Microbiology, 13:857726), causing serious food safety issues. OTA in food and feed can cause import and export trade obstructions, human and animal medical expenses, and result in significant economic losses.

Adopting various means to remove contaminated OTA from agricultural products is an indispensable way to avoid economic losses. At present, detoxification in the food and feed industry is mainly achieved through physical, chemical, and biological methods, among which biological detoxification is safe, efficient, and pollution-free, and has the most application prospects. Biological detoxification methods can be divided into two categories: one is based on the adsorption of microorganisms themselves, and the other is the degradation of OTA by metabolites produced by microorganisms, such as enzymes. There are widely distributed microbial resources that can degrade OTA in nature, and the degradation efficiency of OTA by some bacteria is up to 90%. The degradation efficiency of OTA by Bacillus amyloliquefaciens reached 98.5% within 24 hours (Chang et al., 2015, Food Additives & Contaminants: Part A, 32, 564-571). The degradation efficiency of OTA by Bacillus subtilis, isolated from elk feces, reached 97.6% within 24 hours (Shi, et al., 2014, Journal of the Science of Food and Agriculture, 94:1879-1885). Alcaligenes faecalis can degrade 97% of OTA within 48 hours (Zhang, et al., 2017, Journal of Applied Microbiology, 123:661-668).

At present, the exploration of biological detoxification enzymes and their encoding genes can achieve precise and efficient removal of OTA, improve the stability of detoxification effects, and accumulate key core technologies for biological detoxification methods. At present, there are few reports on detoxification mechanisms and toxin degradation genes, and there is an urgent need to explore new OTA biodegradation enzymes.

### SUMMARY

The purpose of the present invention is to provide a Metarhizium brunneum strain IFST-OT3 and its use in biological detoxification.

Another purpose of the present invention is to provide a detoxification enzyme produced by the Metarhizium brunneum strain IFST-OT3 and its use thereof.

To achieve the purpose of the present invention, in the first aspect, the present invention provides a strain of Metarhizium brunneum IFST-OT3 isolated and purified from soil samples, which has been deposited in the China General Microbiological Culture Collection Center (hereinafter referred to as CGMCC, address: Institute of Microbiology, Chinese Academy of Sciences, No. 3, No. 1 Beichen West Road, Chaoyang District, Beijing 100101), with an accession number of CGMCC No. 40349 and a deposit date of Oct. 28, 2022.

In a second aspect, the present invention provides a microbial agent containing the Metarhizium brunneum strain IFST-OT3.

In a third aspect, the present invention provides the use of the Metarhizium brunneum strain IFST-OT3 or its microbial agent in the detoxification of ochratoxins A and B.

In a fourth aspect, the present invention provides a detoxification enzyme Amh1, produced by the Metarhizium brunneum strain IFST-OT3, where the detoxification enzyme Amh1 includes or consists of the following amino acid sequences:
i) an amino acid sequence as represented by SEQ ID NO:1; or
ii) an amino acid sequence obtained by connecting a tag to a N-terminus and/or C-terminus of i); or
iii) an enzyme having the same function obtained by a substitution, deletion, and/or addition of one or more amino acids in the amino acid sequences of i) or ii).

In a fifth aspect, the present invention provides a nucleic acid molecule encoding the detoxification enzyme Amh1, whose nucleotide sequence is represented by SEQ ID NO:2.

In a sixth aspect, the present invention provides a biological material containing the nucleic acid molecule, where the biological material includes but is not limited to recombinant DNA, expression cassette, transposon, plasmid vector, viral vector, engineered bacterium, or transgenic cell line.

In a seventh aspect, the present invention provides any one of the following uses of the detoxification enzyme Amh1:
1) use in the degradation of ochratoxins A and B;
2) use in the preparation of detoxification agents for ochratoxins A and B.

In an eighth aspect, the present invention provides a method for degrading ochratoxin A, where the detoxification enzyme Amh1 is brought into contact with ochratoxin A in a buffer system at 30 °C to 80 °C and pH 4.0 to 8.0.

Preferably, a concentration of ochratoxin A in the system is 1 to 5 μg/ml.

Preferably, a molar ratio of detoxification enzyme Amh1 to ochratoxin A is 1:130.

A high heterologous expression of the detoxification enzyme in a prokaryotic expression system (Escherichia coli) is achieved in the present invention, and the degradation efficiency for ochratoxin by the detoxification enzyme is extremely high.

By means of the above technical solution, the present invention has at least the following advantages and beneficial effects:

The present invention provides a Metarhizium brunneum strain IFST-OT3 with a strong degrading capability against ochratoxins A and B, and a detoxification enzyme (degradation enzyme) produced by the same, as well as a nucleotide sequence encoding the amidase. Additionally, the optimum physicochemical properties, including temperatures and pH of the enzyme, are provided. The amidase can process OTA toxin with a substrate concentration of 1 μg/ml in a buffer system of 60 °C and pH 6.0, with a degradation efficiency of up to 100% within 3 minutes. The strain IFST-OT3 and the detoxification enzyme produced by the same can be used for the degradation of ochratoxin in agricultural products, and have broad application prospects.

### BRIEF DESCRIPTION OF THE DRAWINGS

FIG. 1 shows HPLC detection charts and HPLC-MS identification of molecular weight of the IFST-OT3 crude enzyme solution when incubated with standards OTA and OTB with a substrate concentration of 1 μg/ml for 24 hours in a preferred Example of the present application. Among them, A and B of FIG. 1 show the HPLC and HPLC-MS detection results of the standard OTA; C and D of FIG. 1 show the HPLC and HPLC-MS detection results of OTA and IFST-OT3 crude enzyme solution incubated for 24 hours; E and F of FIG. 1 show the HPLC and HPLC-MS detection results of standard OTB; and G and H of FIG. 1 show the HPLC and HPLC-MS detection results of OTB and IFST-OT3 crude enzyme solution incubated for 24 hours.

FIG. 2 shows the cloning, prokaryotic expression, and purification of the detoxification enzyme Amh1 gene in a preferred Example of the present invention. Among them, A of FIG. 2 is an agarose gel electrophoresis photo of the Amh gene cloned from a cDNA of the strain IFST-OT3; B of FIG. 2 is a SDS-PAGE electrophoresis photo of Escherichia coli expressed Amh1, with an empty vector expressed as a control; and C of FIG. 2 is a SDS-PAGE electrophoresis photo of purified Amh1.

FIG. 3 shows the degradation efficiency of amidase Amh1 in a preferred Example of the present invention. A of FIG. 3 shows the effect of different pH on the degradation efficiency; B of FIG. 3 shows the effect of different temperatures on the degradation efficiency; C of FIG. 3 shows the effect of different metal ions on the degradation efficiency; and D of FIG. 3 shows the degradation time of 1.0 μg/ml, 2.5 μg/ml and 5.0 μg/ml OTA under the optimum pH 6.0 and optimum temperature (60 °C).

### SPECIFIC MODES FOR CARRYING OUT THE EMBODIMENTS

The following Examples are used to illustrate the present invention, but are not intended to limit the scope of the present invention. Unless otherwise specified, the Examples are carried out under conventional experimental conditions, such as Sambrook et al. Molecular Cloning: a Laboratory Manual (Sambrook J & Russell DW, Molecular Cloning: a Laboratory Manual, 2001), or under the conditions recommended by the manufacturer's instructions.

### Example 1: Isolation and Identification of the Metarhizium brunneum Strain IFST-OT3

Isolation and purification of the Metarhizium brunneum strain IFST-OT3: IFST-OT3 was isolated from farmland soil samples in Beijing. The specific method was as follows: 5 g of soil was taken and 5 mL of sterile water was added, the resultant was shaken on a shaker for 30 min (180 rpm), diluted in gradients of 10, 100, and 1000 times, and spread on PDA medium, respectively. The resultant was inverted and cultured at 28 °C for 72 h, the grown colonies were purified by plate streaking, and the obtained single colony was inoculated on PDA plates for culturing.

On PDA medium, the colony of strain IFST-OT3 appeared fluffy or flocculent, initially white, and then dark green after sporulation, and the edge of the colony was regular and smooth. The hyphae were septate, smooth, and transparent, with 2 to 3 phialides on the conidiophores, and the conidia were unicellular. The strain IFST-OT3 was cultured in PDB medium, and DNA was extracted after 3 days. The EF-1a (primer sequences: 5′-GTACCTCCCAGGCTGACTGC-3′; 5′-GTTCTTGGAGTCACCAGCAACG-3′), RPB2 (5′-GAGGAGCTGAAGAGGACCGG-3′; 5′-AATCGGTGTGTTGGTTCGTCG-3′), and beta-tub (5′-CCGTCCATCAGCTCGTTGAG-3′; 5′-GGAACATGGCAGTGAACTGCTC-3′) genes were amplified and sequenced. The accession numbers of these three genes in NCBI were OP947525, OP947524, and OP947526. All three genes had the highest sequence identity with the sequences of homologous genes in Metarhizium brunneum ARSEF 3297.

Based on the sequencing results and the microbiological characteristics mentioned above, strain IFST-OT3 was identified as Metarhizium brunneum, with an accession number of CGMCC No. 40349.

### Example 2: Degradation Capability of OTA and OTB by Metarhizium brunneum Strain IFST-OT3

At 28 °C, Metarhizium brunneum strain IFST-OT3 was cultured in PDB liquid medium at 180 rpm for 7 days. The collected hyphae were ground into powder using a mortar with continuous addition of liquid nitrogen. 1 g of microbial powder was added to 20 ml of Lysis Buffer (1 mM PMSF, 1% anisole hydrochloride, 10 mM Tris-HCl (pH 7), 150 mM NaCl, 0.5 mM EDTA, 0.01% Triton X-100, and 1 mM DTT), and vortexed to mix well. The resultant was centrifuged at 8000 rpm at 4 °C for 15 min, and the supernatant obtained was the crude enzyme solution. 1 ml of crude enzyme solution was pipetted, and standard OTA or OTB (final concentration of 1 μg/ml) was added. The resultant was incubated at a constant temperature of 28 °C for 24 h, and 1 ml of methanol was added to terminate the reaction. The products were detected by high-performance liquid chromatography (HPLC) and high-performance liquid chromatography tandem mass spectrometry (HPLC-MS).

The results were shown in FIG. 1 (A-H). The peak time of standard OTA was 15.1 min (A of FIG. 1), and its molecular weight identified by mass spectrometry was 403 ([M+H]⁺=404, B of FIG. 1). After OTA and IFST-OT3 were incubated for 24 h, the OTA peak disappeared, and a new peak appeared at 6.2 min (C of FIG. 1), with a molecular weight of 256 ([M−H]⁺=255, D of FIG. 1), and the peak time and molecular weight were consistent with the degradation product OTα after the amide bond of OTA was broken. The peak time of standard OTB was 8.9 min (E of FIG. 1), and its molecular weight identified by mass spectrometry was 369 ([M+H]⁺=370, F of FIG. 1). After OTB and IFST-OT3 were incubated for 24 h, the OTB peak disappeared, and a new peak appeared at 4.6 min (G of FIG. 1), with a molecular weight of 222 ([M−H]⁺=221, H of FIG. 1), and the peak time and molecular weight were consistent with the degradation product OTβ after the amide bond of OTB was broken. The above results showed that Metarhizium brunneum strain IFST-OT3 could degrade OTA and OTB into OTα and OTβ.

### Example 3: Construction of Expression Vector

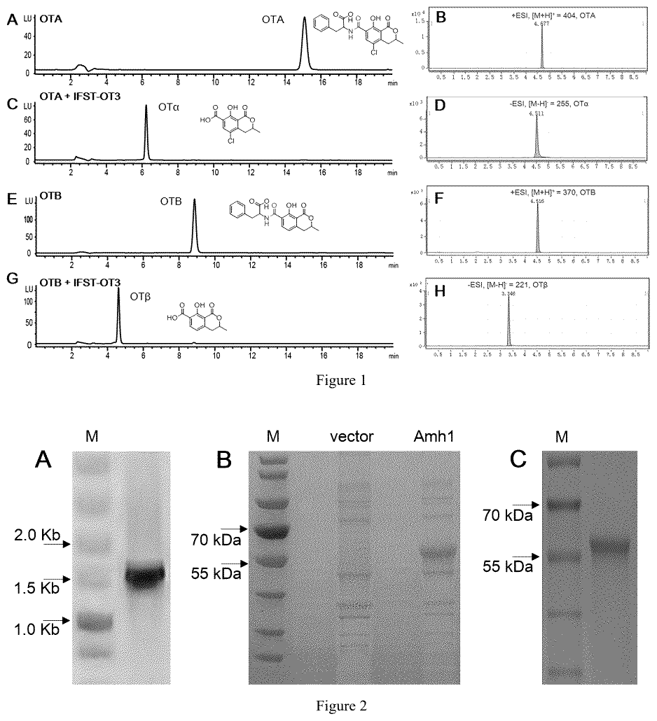

The present application found that Metarhizium brunneum strain IFST-OT3 has the capability to degrade OTA into OTα by extracting fungal crude protein and determining the degradation activity. Through protein isolation and activity determination, mass spectrometry analysis, gene ontology analysis, and protein recombinant expression, an amidase (detoxification enzyme) with degradation activity was finally obtained. Gene Amh1 (GenBank: OP947527) was amplified from reverse-transcribed cDNA of Metarhizium brunneum strain (A of FIG. 2). When designing primers, a BamHI restriction enzyme cutting site was introduced in the upstream primer, and a NotI restriction enzyme cutting site was introduced in the downstream primer. The upstream primer was 5′-GGATCCATGACTAGACGGGTGATTCCCC-3′; and the downstream primer was 5′-GCGGCCGCTCACAACTCCTCTCCCCAAGG-3′. The sequences of restriction endonuclease were underlined. A high-fidelity DNA polymerase with blunt ends was used for product amplification. The target fragment was recovered by gel recovery kit and connected at a ratio of 40 ng with 1 μl of pESAY®-Blun Cloning Vector. The correct sequence of the gene fragment was obtained by the blue-white screening and sequencing. The pET28a (+) vector was linearized by BamHI and NotI restriction endonucleases, and the vector and fragment after restriction digestion were connected at 16 °C for 3 h using DNA Ligation Kit (Mighty Mix, Takara, Japan) to construct a recombinant plasmid. The recombinant plasmid was transformed into E. coli DH5α, and the correct recombinant plasmid expression was obtained by restriction digestion and sequencing verification, and finally, the plasmid was transferred into the expression strain E. coli BL21 (DE3).

### Example 4: Heterologous Expression of Amidase (Detoxification Enzyme) Gene

The transformed strain E. coli BL21 (DE3) monoclone in Example 3 was selected and shaken overnight in 10 mL of LB liquid medium with kanamycin of a final concentration of 80 μg/ml, and it was inoculated into 1 L of LB liquid medium (containing kanamycin) at a 1% inoculum, and shaken at 180 rpm at 37 °C. The bacterial solution concentration OD₆₀₀ was measured, and 1 mg/ml of IPTG solution was added at a ratio of 1/2000 (IPTG final concentration was 1 mM) when the OD value was 0.6 to 0.8. The temperature of the shaker was adjusted to 16 °C after the addition, the bacterial solution was incubated at 100 rpm for about 16 h for induction when it was cooled to 16 °C. 1 L of bacterial solution was centrifuged at 4 °C and 4000 rpm for 10 min, and the supernatant was discarded. The bacteria pellet was dissolved in lysate and stirred for 15 to 20 min (30 mL W1 buffer: 50 mM Tris-HCl pH 8.0, 300 mM NaCl; 1 mM PMSF; 10 mM MgCl₂; 20 μg/mL Lysozyme; and 1 μg/mL DNase I). Homogenization was performed at 4 °C under high pressure (pressurized to 1000 MPa) for 10 to 15 min (depending on the amount of bacteria) until the bacterial solution is clear. The clear bacterial solution was centrifuged at 4 °C and 12000 rpm for 45 min, and the supernatant was kept as the crude enzyme solution. As shown in B of FIG. 2, the SDS-PAGE electrophoresis photo of BL21 expressing Amh1 shows an additional band between 55 kDa and 70 kDa compared with BL21 expressing an empty vector, which is presumed to be the protein expressed by the Amh1 gene.

### Example 5: Protein Purification

The chromatography column with about 1.5 mL of Ni filler was pre-treated. 2 column volumes of Ni-Elute buffer (50 mM Tris-HCl pH 8.0, 300 mM imidazole) were used to rinse, and 2 column volumes of ddH₂O were used to rinse. 1 column volume of W1 buffer in Example 4 was used to rinse, and the supernatant retained in Example 4 was loaded into the chromatography column. To wash away the non-specific binding impurities, 1 column volume of W1 buffer was added to the chromatography column after the sample was drained, and 1 column volume of W2 buffer (50 mM Tris-HCl pH 8.0, 500 mM NaCl, 20 mM imidazole) was added after W1 buffer was drained. In order to wash away the high salt in the purified protein, 1 column volume of W3 buffer (50 mM Tris-HCl pH 8.0) was added after W2 buffer was drained. Finally, Ni-Elute buffer was added to the chromatography column to elute the target protein, and the effluent was collected. The eluate was detected by Bradford until the blue color was lighter.

The purified protein collected in the previous step was concentrated by an anion exchange chromatography column, and then a suitable ultrafiltration tube (using a 30 kDa ultrafiltration tube) was selected for ultrafiltration based on the molecular weight of the protein. After ultrafiltration and concentration, the purified protein was re-concentrated by gel filtration chromatography protein purification method (molecular sieve), and the concentration was determined. The purified protein results were detected by SDS-PAGE electrophoresis, and as shown in C of FIG. 2, the apparent molecular weight of Amh1 protein was consistent with the theoretical molecular weight (55.4 KDa). The purified protein was quickly frozen with liquid nitrogen and stored in a −80 °C freezer for later use.

### Example 6: Enzyme Activity Assay

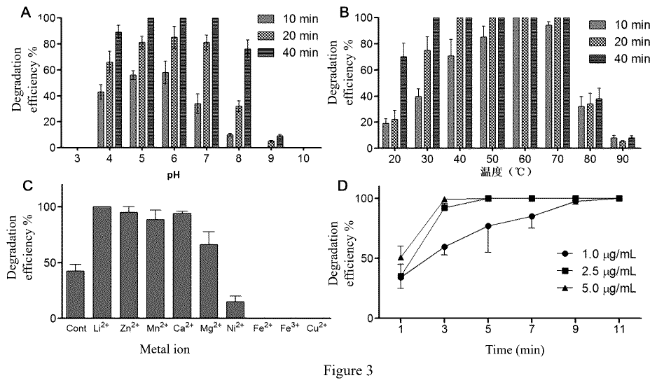

The purified protein Amh1 in Example 5 was diluted to 2.5 μg/mL for enzyme activity assay (the dilution buffer was Tris-HCl pH 6.0). The reaction system was 1 mL, and the purified protein Amh1 at a concentration of 2.5 μg/mL was mixed with 1 μL of 1000 μg/ml standard OTA. The pH gradient was set to 3, 4, 5, 6, 7, 8, 9, 10, and the degradation experiment was carried out at 28 °C. The results showed that the enzyme had a high degradation efficiency for OTA in a slightly acidic environment of pH 4 to 7. OTA could be completely degraded under the condition of pH 5 to 7 after incubating for 40 minutes (A of FIG. 3). The temperature gradient was set to 20 °C, 30 °C, 40 °C, 50 °C, 60 °C, 70 °C, and 80 °C, respectively, and the degradation experiment was carried out under pH 6. OTA was completely degraded under the condition of 30-70 °C after incubating for 40 minutes; OTA was completely degraded under the condition of 40-70 °C after incubating for 20 minutes; and OTA was completely degraded under the condition of 60 °C after incubating for 10 minutes (B of FIG. 3). The above results show that the enzyme can efficiently degrade OTA under the conditions of pH 4 to 7 and 30 °C to 70 °C, exhibiting good acid and heat resistance activity.

In order to further study the effects of nine metal ions on the activity of Amh1, ZnSO₄, MnSO₄, FeSO₄, Fe₂(SO₄)₃, NiSO₄, CuSO₄, MgSO₄, LiCl₂, and CaCl₂) were added to the reaction system to make metal ions at a final concentration of 50 mM. The reaction was carried out at 28 °C and pH 6 for 20 minutes. The results showed that Li²⁺, Zn²⁺, Mn²⁺, and Ca²⁺ significantly increased the enzyme degradation activity, while Ni²⁺ reduced the enzyme activity. In the presence of Fe²⁺, Fe³⁺, and Cu²⁺, Amh1 could not degrade OTA (C of FIG. 3). The degradation efficiency of Amh1 for 1 μg/ml OTA was tested under the conditions of optimum temperature (60 °C) and pH 6.0. The results showed that when the concentration of Amh1 was 1 μg/ml, OTA was completely degraded within 11 minutes; when the concentration was 2.5 μg/ml, OTA was completely degraded within 5 minutes; and when the concentration was 5 μg/ml, OTA was completely degraded within 3 minutes (D of FIG. 3). Compared with other degradation enzymes, Amh1 has a very high degradation efficiency.

Although the present application has been described in detail above with general descriptions and specific embodiments, it is obvious to a person skilled in the art that some modifications or improvements can be made based on the present application. Therefore, these modifications or improvements made without departing from the spirit of the present invention shall all fall within the scope of protection claimed by the present application.

### INDUSTRIAL APPLICABILITY

The present application provides a Metarhizium brunneum strain IFST-OT3 and a detoxification enzyme produced by the same, and its use thereof. The present application provides a Metarhizium brunneum strain IFST-OT3 (Accession number CGMCC No. 40349) with a strong degradation capability against ochratoxins A and B, and a detoxification enzyme (degradation enzyme) Amh1 produced by the same, as well as a nucleotide sequence encoding the amidase. Additionally, the optimum physicochemical properties of the enzyme, including the influence of temperature, pH, and metal ions, are provided. The amidase processes OTA with a substrate concentration of 1 μg/ml in a buffer system at 60 °C and pH 6.0, achieving a degradation efficiency of up to 100% within 3 minutes. Amh1 has strong adaptability to temperature and acidic pH. The strain IFST-OT3 and the detoxification enzyme produced by the same can be used for the degradation of ochratoxins in agricultural products, and have good economic value and application prospects.

1. A Metarhizium brunneum strain IFST-OT3, having an accession number of CGMCC No. 40349.

2. (canceled)

3. (canceled)

4. A detoxification enzyme Amh1 produced by the Metarhizium brunneum strain IFST-OT3 of claim 1, wherein the detoxification enzyme Amh1 comprises or consists of the following amino acid sequences:
i) an amino acid sequence as represented by SEQ ID NO:1; or
ii) an amino acid sequence obtained by connecting a tag to a N-terminus and/or C-terminus of i); or
iii) an enzyme having the same function obtained by a substitution, deletion, and/or addition of one or more amino acids in the amino acid sequences of i) or ii).

5. (canceled)

6. A biological material containing the nucleic acid molecule, wherein the nucleic acid molecule encoding the detoxification enzyme Amh1 of claim 4, and the biological material is recombinant DNA, expression cassette, transposon, plasmid vector, viral vector, engineered bacterium, or transgenic cell line.

7. (canceled)

8. A method for degrading ochratoxin A, wherein the detoxification enzyme Amh1 of claim 4 is brought into contact with ochratoxin A in a buffer system at 30 °C to 80 °C and pH 4.0 to 8.0.

9. The method of claim 8, wherein a concentration of ochratoxin A in the system is 1 to 5 μg/ml.

10. The method of claim 8, wherein a molar ratio of the detoxification enzyme Amh1 to ochratoxin A is 1:130.


SEQUENCE LISTING (2 sequences)

SEQ ID NO: 1 - protein, 485 aa, unidentified
MTRRVIPHSESAESVDDAQHVSRRIYSTGFGIAVRTGHPQSQDDDPEAGVKKAFYTIVTTKLLIPGDGEPIKDAALVVKNKIIDWVGRQADLPNEYTEKPHKLHNVPYLMPGLWDCHVHFAGSNDEREAEEGSTGLSFLADHPTTAGARLARGCWDAIQRGYTSMRDLAGFGCEISKAIEDGVIIGPNIYSAGACLSQLAGHGDVFALPAGDALLNLGVASVKAGQFGAGMSCLVDGVDECRRGVRLQIRRGAKCIKVMASGGVLSRDDNPLYAQFSREELDTIVSEAKRMERTVAAHVHGKPGILQAVEAGVTSVEHVSFADQECIDLIKDRGIIFVGTRTIVNLLLDSKGEGMPKKMWEKAKLVGTHSLEGYKKAIEAGCTIALGTDTEPGFNMAIELQYAVEAGMSSLEAIKAATANGPLTVAGQAPLTGQLKAGYEADMIGVCDNPVEDVKVLQKKSNIGWVWKGGKLFKGPGIGPWGEEL

SEQ ID NO: 2 - DNA, 1458 bp, unidentified
  source 1..1458: mol_type = genomic DNA
atgactagacgggtgattccccattccgaatccgcagagtcggtcgacgacgcgcagcacgtatccaggcgcatctactccacggggtttggcattgccgtccgcacaggacacccgcaatcgcaagacgacgatccggaagcaggggtgaagaaggccttctacacaatcgtgacgacaaagctgctcatcccgggcgacggcgagcctatcaaggatgccgctcttgtagtcaagaacaagattatcgactgggttgggcgacaggccgacctccccaacgagtacaccgagaagcctcataaactgcacaatgtgccttatctgatgcccggcctctgggactgccatgtgcatttcgccggatcaaatgacgagagggaggcagaagagggctcgacgggcctgagctttctcgcagatcaccctactacagctggcgcacgtcttgcaagaggctgctgggacgcaatccaaagaggctacacgtccatgagagaccttgcaggctttggatgcgaaatatccaaggcaatcgaggacggagtcattatcggcccaaacatttacagtgccggagcctgtctcagccagttggctggccacggtgatgtttttgccttgccggccggagacgctcttctgaacctgggggttgcgtcggtcaaggcaggccaattcggagccggtatgagctgcttggtcgatggggttgatgaatgtcgtcggggggtcagactgcagattcgccgcggagcaaagtgcatcaaggtcatggcgtctggcggagtcctgtcgcgcgacgataaccctctatacgcccaattcagccgggaagagctggacacaatcgtgtccgaggcgaagagaatggagaggacggtcgccgcccacgtccacgggaagccgggcatcctccaggccgttgaggcgggcgtgacctcggtcgaacacgtatcattcgctgaccaagagtgtattgatctgattaaggacaggggaatcatatttgtaggcaccaggacgattgtaaacctgcttctcgacagcaagggcgaggggatgcccaagaagatgtgggaaaaggccaagttggttggaacgcactccttggaaggttacaagaaggccatcgaagcaggctgcacgattgcgcttgggaccgataccgagcccggattcaacatggccattgagcttcaatatgctgtcgaggccggcatgtccagcttggaggctatcaaggctgccactgccaacgggccattgactgttgctgggcaggctcctctgacgggccaactaaaggccggctatgaggctgacatgattggtgtttgtgacaatccagtagaggatgtgaaagtgctgcagaagaagagcaatattggatgggtatggaagggcggaaagctgtttaaaggtccgggaattggtccttggggagaggagttgtga